(19) United States
(12) Patent Application Publication
(10) Pub. No.: US 2017/0312216 A1
(43) Pub. Date: Nov. 2, 2017

(54) COSMETIC COMPOSITION FOR IMPROVING ANTI-OXIDATION, ANTI-INFLAMMATION AND ATOPIC SKIN BY USING FERMENTED BLACK TEA LIQUID AS EFFECTIVE INGREDIENT AND METHOD OF PREPARING THE SAME

(71) Applicants: Danjoungbio Co., Ltd., Wonju-si (KR); The Trumetic Co., Ltd., Seoul (KR)

(72) Inventors: Duggu KIM, Anyang-si (KR); EUI SU CHOUNG, Wonju-si (KR); KYU RAN LEE, Wonju-si (KR); JAE UNG YUN, Wonju-si (KR); MIN HEE PARK, Wonju-si (KR); JONG SUB LEE, Wonju-si (KR); HAE BYOUNG BAE, Wonju-si (KR); YOON KI CHANG, Centreville, VA (US)

(73) Assignee: Danjoungbio Co., Ltd., Wonju-si (KR)

(21) Appl. No.: 15/522,697

(22) PCT Filed: Dec. 2, 2015
(86) PCT No.: PCT/US15/63490

§ 371 Date: Apr. 27, 2017

(30) Foreign Application Priority Data
Oct. 27, 2014 (KR) 10-2014-0146415

(51) Int. Cl. A61K 8/9789; A61Q 19/00

(52) U.S. Cl. CPC A61K 8/9789; A61Q 19/005; A61K 2800/782; A61K 2800/75; A61K 2236/13; A61K 2236/19; A61K 2236/17; A61K 2236/15; A61K 2800/85; A61K 2236/331

(57) ABSTRACT

Disclosed are a cosmetic composition for improving anti-oxidation, anti-inflammation and atopic skin by using fermented liquid of black tea as an effective ingredient and a method of preparing the same. The cosmetic composition includes the fermented black tea liquid obtained by primarily fermenting black tea with saccharomyces, secondarily fermenting the black tea with gluconacetobacter and tertiarily fermenting the black tea with bacillus.

### CROSS-REFERENCE TO RELATED APPLICATION

This application claims the benefit of Korean application No. 10-2014-0146415, filed on Oct. 27, 2014 with the Korean Intellectual Property Office, the disclosure of which is incorporated herein by reference.

### TECHNICAL FIELD

The present invention relates to a cosmetic composition for improving anti-oxidation, anti-inflammation and atopic skin by using fermented liquid of black tea as an effective ingredient and a method of preparing the same, and more specifically, to cosmetic composition for improving anti-oxidation, anti-inflammation and atopic skin by using fermented liquid of black tea or extract extracted from the fermented liquid as an effective ingredient which is obtained by primarily fermenting black tea with saccharomyces, secondarily fermenting black tea with gluconacetobacter or thirdly fermenting black tea with bacillus, that the cosmetic composition has excellent effect in anti-oxidation, anti-inflammation and atopic skin improvement and has no side effects such as skin stimulation, and a method of preparing the same.

In addition, the present invention relates to a cosmetic composition for improving anti-oxidation, anti-inflammation and atopic skin by using fermented liquid, of black tea as an effective ingredient which inhibits lipoxygenase and hyaluronidase and removes free radical so that anti-oxidation, anti-inflammation and atopic skin may be improved, and a method of preparing the same.

### BACKGROUND ART

When skin directly exposed to external environment is excessively exposed to ultraviolet rays or contaminants, skin irritant such as erythema, edema or pruritus and an inflammatory response are caused. It has been known that skin trouble has a bad effect on the appearance and in addition, a material generated during inflammatory response causes pigmentation to promote the collapse of skin elastic fiber so that wrinkles are increased. In addition, it has been known that skin damage is concerned with free radical.

The free radical theory had been proposed in the middle of the 1950's by D. Barman and has been recently spotlighted in the related society and industry. The free radical destroys cells, or cuts off cross-links connective tissue of skin dermis, so that troubles such as wrinkles, skin cancer, skin killing, rheumatoid arthritis, atopic dermatitis and acne are caused.

As the causes of the generation of the free radical, leucocyte phagocytosis, electron transport system during ATP generation of mitochondria, myeloper oxide (MPO) reaction, ultraviolet rays, tobacco, a normal metabolic process, stress, pollutant and bacteria are known.

The phagocytosis is a process by which a cell body receives a foreign substance or bacteria to resolve it to be harmless.

Although a human body has antioxidant (radical scavenger) such as superoxide dismutase (SDD), catalase, vitamin E, vitamin C, or uviquinol, an antioxidant system is incapacitated due to age, pollution, ultraviolet rays or stress, so that free radical is gradually increased.

The increased free radical destroys collagen and elastin hyaluronic acid constituting connective tissue of skin dermis, so that the settling of skin (wrinkle) is partially caused. In addition, the lipid of cell membrane is oxidized to destroy cells, so that disease such as dermatitis, acne or skin cancer is caused.

During the generation of melanin, the free radical is concerned with self oxidation reaction (dopaquinone→melanin), so that the free radical may casuse melisma, freckle or wrinkles (Korean Cosmetic Society, volume 23, No. 1, pages 75˜132) .

Inflammation is a physiological reaction for protecting a body from being invaded by a foreign substance such bacteria or being mechanically damaged in harmful environment. The inflammation causes various kinds of polymerpho-nuclear leukocytes (PMHs) and immunity substance to be increased, so that the cells secrete various kinds of proteases and cytokine which are products of inflammation cells, thereby performing therapy and protection. The enzymes such as elastase, hyalurinxdase and lipoxygenase nave been, well known as proteases or lipolytic enzymes.

Meanwhile, the reactions of enzymes may cause adjacent tissue cells and non-tissue cells to be harmfully damaged, so that serious tissue damage such as chronic inflammation may be caused.

As described above, the hyper inflammation causes cells and connective tissue to be damaged due to the protease and the damage of the connective tissue reduces the skin elasticity to cause wrinkles. In addition, the hyper inflammation has a bad effect on cell regeneration and proliferation to cause skin aging to be accelerated, so that partial erythema and swell are caused on skin, if the connective tissue is seriously damaged and inflammation reaction is continuous, it is very difficult to normally recover the tissue and function of skin.

The generation of mediums increasing inflammation and leukocyte is implemented through a process by arachidonic acid. This process includes a process by cyclooxygenase and a process by lipoxygenase, and leukotriene and prostagladine generated by the enzymes act as inflammatory mediators. Thus, the leukotriene and prostagladine inhibitors prevent an inflammatory mediator from being generated and in addition, act as an anti-inflammatory material, which prevents cell membrane from being damaged due to the free radical generated during inflammation and prohibits the peroxidation of tissue. In addition, the protein and lipolytic enzymes generated due to hyper inflammation is inhibited, so that the damaged cells is protected, thereby inhibiting skin from, being aged and improving atopic skin.

The related art used currently and generally and having an anti-oxidation effect had been disclosed in Korean Registered Patent No. 10-1315325 entitled “Skin external composition containing anti-oxidation components for improving skin wrinkles and whitening skin”. The skin external composition contains at least two anti-oxidant ingredients selected from the group consisting of lipoic acid, tocopherol, coenzyme, Q10, selenium and vitamin C, such that the activation, of protection factors in skin associated with removal of free radical and anti-oxidation is increased, thereby improving skin wrinkles and whitening skin.

The related art had been used for preventing wrinkles and skin diseases by mixing the skin external composition as a material having a free radical removal effect with cosmetics or a medicine. However, the composition is expensive and the mixture is not stable, so that it is difficult to obtain a practical effect.

There is necessarily required to develop a material having effects of anti-oxidation, anti-inflammation and relieving of skin irritation in order to relieve skin irritation and inflammation, due to various kinds of stresses and reduce harmful side effect due to using as cosmetics. In addition, there is required a method of effectively extracting useful, components from a natural product which has a small aftereffect on human body while minimizing destruction of the useful components.

Thus, there is a need to develop a natural extract having excellent effect on anti-oxidation, anti-inflammation and atopic skin in order to improve skin trouble due to free radical and inflammation and no aftereffect, cosmetics contain the extract and an optimal extracting method.

### DISCLOSURE

### Technical Problem

To solve the problems described above, an object of the present invention is to provide a cosmetic composition for improving anti-oxidation, anti-inflammation and atopic skin by using fermented liquid of black tea as an effective ingredient which is obtained by primarily fermenting black tea with saccharomyces, secondarily fermenting black tea with gluconacetobacter or thirdly fermenting black tea with bacillus, or extracted from them, so that the cosmetic composition has excellent effect in anti-oxidation, anti-inflammation and atopic skin improvement and has no side effects such as skin stimulation, and a method of preparing the same.

Another object of the present invention is to provide a cosmetic composition for improving anti-oxidation, anti-inflammation and atopic skin by using fermented liquid of black tea as an effective ingredient which inhibits lipoxygenase and hyaluronidase and removes free radical so that anti-oxidation, anti-inflammation and atopic skin may be improved, and a method of preparing the same.

### Technical Solution

According to the present invention, there is provided a cosmetic composition for improving anti-oxidation, anti-inflammation and atopic skin, which contains fermented liquid of black tea obtained by primarily fermenting black tea with saccharomyces, secondarily fermenting black tea with gluconacetobacter or thirdly fermenting black tea with bacillus.

In addition, according to the present invention, there is provided a cosmetic composition for improving anti-oxidation, anti-inflammation and atopic skin, which contains extract of a fermented material of black tea obtained by primarily fermenting black tea with saccharomyces, secondarily fermenting black tea with gluconacetobacter or thirdly fermenting black tea with bacillus.

Preferably, the black tea is extracted by using one extract solvent selected from the group consisting of water, anhydrous or hydrous lower alcohol of hydrocarbon 1 to 4, acetone, ethyl acetate, butyl acetate, and 1,3-butylene glycol.

In addition, according to the present invention, there is provided a method of preparing a cosmetic composition for improving anti-oxidation, anti-inflammation and atopic skin by using fermented black tea liquid as an effective ingredient, which includes; obtaining an extract of black tea (S10); obtaining primary fermented liquid by inoculating saccharomyces as strain on the extract of the black tea (S20); obtaining secondary fermented liquid by inoculating gluconacetobacter as strain on the primary fermented liquid (S30); obtaining tertiary fermented liquid, by inoculating bacillus as strain on the secondary fermented liquid (S40); obtaining an extract of the fermented black tea liquid, through a hot-water extracting scheme after removing cultured bacteria from the tertiary fermented liquid (S50); and obtaining the cosmetic composition by further adding stabiliser, vitamin, pigment, dye and perfume to the fermented black tea liquid of step S40 or to the extract of the fermented black tea liquid of step (S60).

### ADVANTAGEOUS EFFECTS

According to the present invention described above, the fermented liquid of black tea or extract extracted from the fermented liquid, which is obtained by primarily fermenting black tea with saccharomyces, secondarily fermenting black tea with gluconacetobacter or thirdly fermenting black tea with bacillus, has an excellent effect in anti-oxidation, anti-inflammation and atopic skin improvement and has no side effects such as skin stimulation.

In addition, according to the present invention, there are provided a cosmetic composition for improving anti-oxidation, anti-inflammation and atopic skin by using fermented liquid of black tea as an effective ingredient, and a method of preparing the same, where the cosmetic composition inhibits lipoxygenase and hyaluronidase and removes free radical,

### DESCRIPTION OF DRAWINGS

FIG. 1 is a flowchart illustrating a method of preparing a cosmetic composition for improving anti-oxidation, anti-inflammation and atopic skin by using fermented liquid of black tea as an effective ingredient according to an embodiment of the present invention,

### BEST MODE

### Mode for Invention

The present invention relates to a study of protecting operation of skin from oxidation, an inflammatory reaction and an atopic symptom based on a fermenting technique of maximizing anti-oxidation effect and effectiveness. As the result, the present invention has been completed by confirming that the fermented liquid, which is obtained by primarily fermenting black tea with saccharomyces, secondarily fermenting black tea with gluconacetobacter or thirdly fermenting black tea with bacillus, has an effect of greatly improving anti-oxidation, anti-inflammation and atopic skin.

Hereinafter, a cosmetic composition for improving anti-oxidation, anti-inflammation and atopic skin by using fermented liquid of black tea as an effective ingredient according to the present invention will be described as follows.

The present invention provides a cosmetic composition for improving anti-oxidation, anti-inflammation and atopic skin, which contains fermented liquid of black tea obtained by primarily fermenting black tea with saccharomyces, secondarily fermenting black tea with gluconacetobacter or thirdly fermenting black, tea with bacillus.

In addition, the present invention provides a cosmetic composition for improving anti-oxidation, anti-inflammation and atopic skin, which contains extract, of a fermented material of black tea obtained by primarily fermenting black tea with saccharomyces, secondarily fermenting black tea with gluconacetobacter or thirdly fermenting black tea with bacillus.

The black tea is a kind of tea (camellia sinensis) more fermented than white, green and oolong teas. The black tea is stronger in flavor and contains more caffeine than the teas.

The tea is called “red tea” in the East because the tea liquid is red, but in the West, the tea is called black tea because the tea leaves are black. In the West, the red tea more commonly refers to rooibos tea of South Africa.

While green tea usually loses its flavor within a year, black tea retains its flavor for several years. For this reason, it has long been an article of trade, and compressed bricks of black tea even served as a form of de facto currency in Mongolia, Tibet and Siberia into the 19th century. Although this is description about tea classified as pure tea, westerners thought pure tea as black tea drunk by them. Referring to the documents written by westerners, it is confirmed that tea served as a form of de facto currency.

Traditionally, only black tea had been known to the West. Although green tea has been widely populated, the black tea still is 90% or more of the total teas sold in the West,

A preferable dry weight ratio of black tea is in the range of 10 to 40 weight% of black tea. Preferably, the black tea is extracted by using one extract solvent selected from the group consisting of water, anhydrous or hydrous lower alcohol of hydrocarbon 1 to 4, acetone, ethyl acetate, butyl acetate, and 1,3-butylens glycol.

The black tea may be prepared according to a conventional scheme well known in the art. That is, black tea extract may be prepared by using a conventional solvent under conventional conditions of temperature and pressure.

For example, the black tea, which is an effective ingredient of a composition according to the present invention, may be extracted by using a solvent selected from the group consisting of water, anhydrous or hydrous lower alcohol of hydrocarbon 1 to 4, acetone, ethyl acetate, butyl acetate, and 1,3-butylene glycol, or may be prepared by mixing extract after the black tea is extracted by using a solvent.

Alcohol is good as the solvent. Ethanol is preferable as the solvent. Ethanol of 70% is more preferable as the solvent. A suitable amount of added solvent is 1 to 30 times of a dry weight of black tea, preferably, 10 to 30 times, more preferably 20 times.

Meanwhile, the fermented liquid, which is obtained by primarily fermenting black tea with saccharomyces, secondarily fermenting black tea with gluconacetobacter or thirdly fermenting black tea with bacillus according to the present invention, includes the extract through a conventional purifying process as well as the extract by the above-described extracting scheme,

For example, it is understood that fractions obtained through additionally performed various schemes such as separation using a ultrafiltration membrane a molecular weight cut-off value, separation using various chromatography (prepared for the purpose of separation according to a size, electric charges, hydrophobic or affinity) are included in the fermented liquid, which is obtained by primarily fermenting black tea with saccharomyces, secondarily fermenting black tea with gluconacetobacter or thirdly fermenting black tea with bacillus according to the present invention.

In addition, the fermented liquid, which is obtained by primarily fermenting black tea with saccharomyces, secondarily fermenting black tea with gluconacetobacter or thirdly fermenting black tea with bacillus according to the present invention, includes a fermented material prepared in a powder state through an additional process such as vacuum distillation, freeze drying or spray drying.

The black tea fermented liquid or extract, which is obtained by primarily fermenting black tea with saccharomyces, secondarily fermenting black tea with gluconacetobacter or thirdly fermenting black tea with bacillus according to the present invention, is a natural safe medicine having an excellent effect in anti-oxidation, anti-inflammation and atopic skin improvement without any side effects such as skin stimulation,, and the preparing cost is inexpensive,

The fermented liquid, which is obtained by primarily fermenting black tea with saccharomyces, secondarily fermenting black tea with gluconacetobacter or thirdly fermenting black tea with bacillus according to the present invention, may be in a liquefied state or dried into power.

The saccharomyces, which is 1 genus of yeast included in ascomycetes and multilateral budding ascosporogenous yeast having an egg shape or an elliptical shape, includes one or at least two strains selected from the group consisting of saccharomyces bayanus, saccharomyces boulardii, saccharomyces cerevisiae, saccharomyces chevalieri, saccharomyces dairenensis, saccharomyces ellipsoideus, saccharomyces eubayanus, saccharomyces exiguus, saccharomyces florentinus and saccharomyces uvarum.

The saccharmyces is preferably saccharomyces cerevisiae. The saccharomyces cerevisiae, which is typical yeast included in ascomycetes, includes baker yeast, brewery yeast and yeast, used in brewing.

The gluconacetobacter, which is acetic acid bacteria excellent in acetic acid fermentation and cellulose synthesis, is one or at least two strains selected from the group consisting of gluconacetobacter hansenii, gluconacetobacter intermedius, gluconacetobacter sacchari and gluconacetobacter xylinum.

Preferably, the gluconacetobacter is gluconacetobacter xylinum.

The bacillus, which is one genus of family bacillaceae of schizomycetes, has a bar shape, is gram positive and is aerobic bacteria making spore, is one or at least two strains selected from the group consisting of bacillus cereus, bacillus subtilis, bacillus licheniformis, bacillus megateriums, bacillus natto, bacillus citreus, bacillus circulans, bacillus mesentricus and bacillus pumilus.

Preferable, the bacillus is bacillus cereus which is bacterial positive in gram staining. The bacillus cereus is aerobic, forms spore and most exists in soil.

The cosmetic composition, for improving anti-oxidation, anti-inflammation and atopic skin by using fermented liquid of black tea as an effective ingredient according to the present invention has effects on anti-inflamination and anti-oxidation and is very stable in applied products. In addition, the cosmetic composition inhibits lipoxygenase and hyaluronidase activations, removes free radical, and relieves skin irritation and inflammation.

According to a preferable embodiment of the present invention, the content of the black tea fermented liquid obtained by primarily fermenting black tea with saccharomyces, secondarily fermenting black tea with gluconacetobacter or thirdly fermenting black tea with bacillus or the content of the extract extracted from the fermented liquid is in the range of 0.01 to 20.0% by weight based on the total weight of the cosmetic composition, preferably, is in the range of 0.1 to 15.0% by weight based on the total weight of the cosmetic composition, or more preferably, in the range of 1% by weight to 10.0 % by weight the total weight of the cosmetic composition.

When the black tea fermented liquid or the extract extracted from the fermented liquid is less than 0.01% by weight based on the total weight of the cosmetic composition, the lipoxygenase and hyaluronidase activations are not inhibited and free radical is not removed. When the black tea fermented liquid or the extract extracted from the fermented liquid exceeds 20% by weight based on the total weight of the cosmetic composition, dry skin may be itchy or tingled.

The black tea fermented liquid or the extract extracted from the fermented liquid according to the present invention has an effect in the anti-oxidation improvement, the anti-inflammation improvement and atopic skin improvement has no skin irritation aftereffect except for a person having dry skin, so that the black tea fermented liquid of 100% or the extract of 100% extracted from the fermented liquid may be used as a cosmetic composition.

A component included in the cosmetic composition of the present invention includes components, such as adjuvant including antioxidant, solubilizer, stabilizer, vitamin, paint, pigment and perfume, or a supporting material, conventionally used for a cosmetic composition as well as the fermented liquid of black tea as an effective ingredient which is obtained by primarily fermenting black tea with saccharomyces, secondarily fermenting black tea with gluconacetobacter or thirdly fermenting black tea with bacillus.

The cosmetic composition according to the present invention may be prepared into formulations conventionally prepared in the art. For example, the cosmetic composition according to the present invention may be formulated into solution, suspension, emulsion, paste, gel, cream, lotion, powder, soap, surface active agent containing cleansing, oil, powder foundation, emulsion foundation, wax foundation, and spray, but the present invention is not limited thereto. In more detail, the cosmetic composition according to the present invention may be formulated into skin soft cosmetic water, nutrition cream, massage cream, essence, pack, eye cream, cleansing cream, cleansing foam, cleansing water, spray or power.

When the formulation of the present invention is paste, cream or gel, at least one selected from animal oil, vegetable oil, wax, paraffin, starch, tragacanth, cellulose derivatives, polyethylene glycol, silicon, bentonite, silica, talc and zinc oxide may be used as a supporting component.

When the formulation of the present invention is powder or spray, lactose, talc, silica, aluminum hydroxyl, calcium silicate, or polyamide powder may be used as a supporting component. Specifically, when the formulation of the present invention is spray, the supporting component may include propellant such as fluorohydrocarbon, propane/butane or dimethyl ether.

When the formulation of the present invention is solution or emulsion, solvent, solubilizer, or emulsion agent is used as a supporting component. For example, as a supporting component, at least one selected from water, ethanol, isopropanol, ethylene carbonate, ethyl acetate, benzyl alcohol, benzyl benzoate, propylene glycol, butylene glycol, 1,3-butylene glycol oil, polyoxyethylene hydrogenated caster oil, glycerol, glycerin, aliphatic copolyesters, phenoxyethanol, triethanolamine, polyethylene glycol, bees wax, polysorbate 60, sorbitan sesquioleate, paraffin, sorbitan stearate, lipophilic glyceryl monostearate, stearic acid, glyceryl stearate/PEG-400stearate, carboxypolymer, sitosterol, polygyceryl 2-olate, ceramide, cholesterola steareth-4, dicetylphosphate, macadamia oil, carboxyvinylpolymer, xanthan gum and sorbitan fatty acid ester may be used.

When the formulation of the present invention is suspension, as a supporting component, at least one selected from liquid diluent such as water, ethanol, glycerin, butylene glycol or propylene glycol, suspension such as ethoxylated isostearyl alcohol, polyoxyethlene sorbitol ester, or polyoxyethlene sorbitan ester, microcrystalline cellulose, hydroxyethyl cellulose, sodium hyaluronate, phenoxyethanol, aluminium metahydroxides, bentonite, stearic acid, cetyl alcohol, glyceryl monostearate, polyoxyethylene sorbitan monostearate, sorbitan sesquioleate, glyceryl monostearate/glyceryl stearate/polyoxyethylene stearate, wax, paraffin, squalane, caprylic/capric triglycerides, carboxyvinylpolymer, triethanolamine, agar and tragacanth may be used.

When the formulation of the present invention is surface active agent containing cleansing, as a supporting component, at least one selected from aliphatic alcohol sulfate, aliphatic alcohol ether sulfate, sulfosuccinate acid monoester, isethionate, imidazolinium derivatives, methyl tartrate, sarcoslnates, fatty acid amid ether sulfate, alkylamidobetaine, aliphatic alcohol, fatty acid glyceride, fatty acid diethanolamide, vegetable oil, lanolin derivatives and ethoxylated glycerol fatty acid ester may be used.

Hereinafter, a method, of preparing a cosmetic composition for improving anti-oxidation, anti-inflammation and atopic skin by using fermented liquid of black tea as an effective ingredient according to the present invention will be described with reference to accompanying drawings.

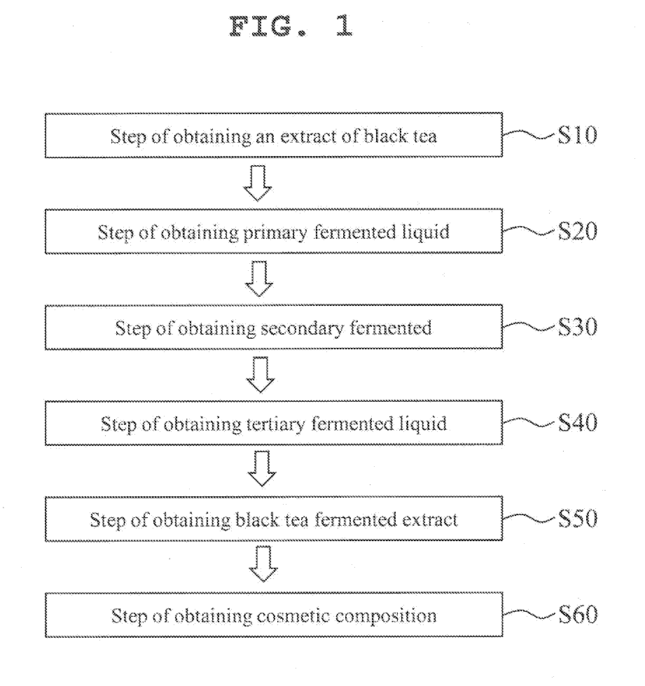

FIG. 1 is a flowchart illustrating a method of preparing a cosmetic composition for improving anti-oxidation, anti-inflammation and atopic skin by using fermented liquid of black tea as an effective ingredient according to an embodiment of the present invention.

Hereinafter, a preferable embodiment according to the present invention will be described with reference to the accompanying drawing. It is preferable that a method of preparing a cosmetic composition for improving anti-oxidation, anti-inflammation and atopic skin by using fermented liquid of black tea as an effective ingredient according to the present invention includes following steps, but the present invention is not limited thereto.

A method according to present invention includes the steps of: obtaining the fermented liquid of black tea (S10); obtaining primary fermented liquid by inoculating saccharomyces as strain on the fermented liquid (S20); obtaining secondary fermented liquid by inoculating gluconacetobacter as strain on the primary fermented liquid (S30); obtaining third fermented liquid by inoculating bacillus as strain on the secondary fermented liquid (S40); obtaining extract of the fermented material of black tea through a hot-water extracting scheme after removing cultured bacteria from the third fermented liquid (S50); and obtaining the cosmetic composition by further adding stabilizer, vitamin., paint, pigment and perfume to the fermented material of black tea in the obtaining of the third fermented liquid or the extract of the fermented material of black tea in the obtaining of the extract (S60).

Each step of the preparing method according to the present invention will be described as follows.

Step S10 of obtaining the fermented liquid of black tea is performed.

After the black tea is cleaned with cleaning water and dried, all the black tea is pulverized. Then, fine pulverized black tea is obtained by using a strainer of 50 to 200 meshes. After the distilled water of 100 to 300 g/L is added to the pulverized black tea, reflux extraction and macerating operation is performed three times for three or seven hours under a bath condition. Then, the extract is prepared through filtering by a filter of 3 to 8 μm and is used for the fermenting process.

Step S20 of obtaining the primary fermented liquid by inoculating saccharmyces as strain on the black tea fermented liquid is performed.

After the saccharmyces as strain, and an YM broth medium are added to the black tea extract obtained in the step S10, the black tea extract is fermented for 80 to 150 hours under the expiration condition of a temperature in the range of 20° C to 25° C. After cultivation, a culture medium is centrifuged to primarily remove cultured bacteria and sterilized to be prevented from, being cultured any more.

Step S30 of obtaining secondary fermented liquid by inoculating gluconacetobacter as strain on the primary fermented liquid is performed.

After the gluconacetobacter as strain and a mannitol broth medium are added to the primary fermented liquid obtained in the step S20, the primary fermented liquid is fermented for 80to 150 hours under the expiration condition of a temperature in the range of 22° C., to 28° C. After cultivation, a culture medium is centrifuged to primarily remove cultured bacteria and sterilized to be prevented from being cultured any more.

Step S40 of obtaining the third fermented liquid by inoculating bacillus as strain on the secondary fermented liquid is performed.

After pH of the secondary fermented liquid obtained in the step S30 is adjusted and the bacillus as strain and a nutrient broth medium are added to the secondary fermented liquid, the secondary fermented liquid is fermented for 60 to 120 hours under the expiration condition of a temperature in the range of 25° C. to 33° C.

The content of the third fermented material of black tea obtained in the step S40 may be in the range of 0.01 to 20.0% by weight based on a total weight of the cosmetic composition

When the black tea third fermented liquid is less than 0.01% by weight based on the total weight of the cosmetic composition, the lipoxygenase, and hyaluronidase activations are not inhibited and free radical, is not removed. When the black tea fermented liquid exceeds 20% by weight based on the total weight of the cosmetic: composition, dry skin may be itchy or tingled.

Step S50 of obtaining black tea fermented extract through a hot-water extracting scheme after removing cultured bacteria from the third fermented liquid is performed.

The third fermented liquid is sterilized and antiseptic is added to the third fermented liquid, the black tea fermented extract is obtained through the hot-water extracting scheme. After ethanol is added to the black tea fermented liquid from which the cultured bacteria is removed sterilized in order to finally obtain ethanol solution of 70 % (V/V), reflux extraction and macerating operation is performed three times for three or seven hours. Then, the black tea fermented extract is filtered by a filter of 3 to 8 so that the black tea fermented

extract is obtained.

The content of the black tea fermented extract obtained through the step S50 may be in the range of 0.01 to 20.0% by weight based on a total weight of the cosmetic composition.

When the black tea fermented extract is less than 0.01% by weight based on the total weight of the cosmetic composition, the lipoxygenase and hyaluronidase activations are not inhibited and free radical is not removed. When the black, tea fermented extract exceeds 20% by weight based on the total weight of the cosmetic composition, dry skin may be itchy or tingled,

In step S60, the cosmetic composition is obtained by adding stabilizer, vitamin, paint, pigment and perfume to the black tea fermented liquid obtained in the step S40 or the black; tea fermented extract, obtained in the step S50. Hereinafter, a preparing example, an embodiment, a comparative example, and a formulation example according to the present invention will be described in detail.

The preparing example, embodiment, comparative example, and formulation example are only examples of the present invention, and the present invention is not limited thereto.

### PREPARING EXAMPLE

Method of Preparing Black Tea Extract

After the black tea was cleaned with cleaning water and dried, ail the black tea was pulverized. Then, fine pulverized black tea was obtained by using a strainer of 100 meshes. After the distilled water of 150 g/L was added to the pulverized black tea, reflux extraction and macerating operation was performed three times for five hours under a bath condition. Then, the extract was prepared through filtering by a filter of 6 μm (whatman #3) and was used for the fermenting process.

In addition the fermented liquid was vacuum-concentrated and freeze-dried to be used for evaluation of efficacy at 50° C. or less.

Embodiment

Method of Preparing Black Tea Fermented Liquid and Black Tea Fermented Extract

After the saccharomyces cerevisiae (KCTC 7917) as strain and an YM broth medium were added to the black tea extract prepared in the preparing example, the black tea extract was primarily fermented for 120 hours under the expiration condition of 24° C. After cultivation, a culture medium was centrifuged to primarily remove cultured bacteria and sterilized to be prevented from being cultured any more (121° C., quarter-hour, 1.5 atm).

After gluconacetobacter xylinus subsp. xylinus (KCCM 40216) and a marmitol broth medium were added to the primary fermented liquid for secondary fermentation, the primary fermented liquid was secondarily fermented for 96 hours under the expiration condition of 26° C. After cultivation, a culture medium was centrifuged to primarily remove cultured bacteria and sterilized to be prevented from being cultured any more (121° C., quarter-hour, 1.5 atm).

After pH of the secondary fermented liquid was adjusted for third fermentation and Bacillus cereus (KCTC 1661) and a nutrient broth medium were added to the secondary fermented liquid, the secondary fermented liquid was fermented for 72 hours at 30° C.

After the third fermented liquid was sterilized and preservatives were supplied, the third fermented liquid was filtered to prepare the black tea fermented liquid.

After fermentation, ethanol is added to the black tea fermented liquid from which the cultured bacteria is primarily removed in order to finally obtain ethanol solution of 70% (V/V), and reflux extraction and macerating operation is performed three times for five hours. Then, the extract was prepared through filtering by a filter of 6 μm (whatman #3). The filtered extract was transferred into a concentrating vessel to be vacuum-concentrated and freeze-dried at 50° C. or less.

### Comparative Example 1

Comparative Example 1 of a Method of Preparing Black Tea Pine Mushroom Fermented Liquid Containing Pine Mushroom Mycelium

According to comparative example 1, the black tea pine mushroom fermented liquid was prepared by using the black tea extract obtained in preparing example 1 as pine mushroom mycelium. Except for the pine mushroom, the remaining experiment method was the same as embodiment 1.

### Comparative Example 2

Comparative Example 1 of a Method of Preparation Black Tea Pine Mushroom Fermented Liquid Containing Sarcodon Aspratus Mycelium

According to comparative example 2, the black tea sarcodon aspratus fermented liquid was prepared by using the black tea extract obtained in preparing example 2 as sarcodon aspratus mycelium. Except for the sarcodon aspratus, the remaining experiment, method was the same as embodiment 1.

### Experiment Example 1

Cell Proliferation and Toxicity Confirmation by Black Tea Third Fermented Liquid of the Present Invention

It was confirmed that the dried powder of the black tea fermented liquid prepared in embodiment 1, and comparative examples 1 and 2 and the dried powder prepared in preparing example 1 have an effect on cell proliferation.

An experiment for confirming cell proliferation and toxicity was performed in a following method.

The proliferating cell 3T3 (fibroblast cell line) was divided into 5,000 cell/wall in 96 wall micro-plate and proliferated in a thermostat for 30 minutes. Then, samples were supplied at concentrations of 0.01, 0.05, 0.1 and 0.5%, W/V, respectively and proliferated for 72 hours. After 72 hours, thiazoline blue was supplied and further proliferated for 4 hours. After the culture medium was discarded, reaction termination liquid was supplied into each wail of the micro-plate and agitated for 5 minutes. Absorbance was measured at 570 nm. A fetal bovine serum (PBS) medium of 10% was supplied to a control group as much as an amount of introduced samples and the control group was simultaneously proliferated under an optimal condition of cell proliferation. A cell proliferation rate of the sample introducing control group was calculated assuming that the cell proliferation of the control group is 100%.

The cell proliferation effect was calculated through formula 1 and the result is shown In following table 1.

$$Cell Proliferation Effect ( \% ) = \frac{\begin{matrix}Absorbance In Treating Extract - \\ Absorbance of Control Group\end{matrix}}{Absorbance o f Control Group} \times 100 \qquad [ Formula 1 ]$$

TABLE 1
Effect of cell proliferation and toxicity relaxation by
black tea fermented liquid

| | Cell proliferation effect (%) | | | |
|---|---|---|---|---|
| Concentration | | Comparative example | | Preparing |
| (%) | Embodiment | 1 | 2 | example |
| Control | 0 | 0 | 0 | 0 |
| group | | | | |
| 0.01 | 8.2 | 5.1 | 6.2 | 1.9 |
| 0.05 | 16.3 | 8.8 | 8.1 | 3.5 |
| 0.1 | 21.5 | 15.1 | 13.9 | 7.1 |
| 0.5 | 30.7 | 18.2 | 17.6 | 11.3 |

As shown in Table 1, it was confirmed that, when it; was assumed that the cell proliferation under an optimal cell proliferation condition is 100%, the black tea fermented liquid prepared in the embodiment and comparative examples 1 and 2 and the black tea fermented extract prepared in preparing example 1 had no cell toxicity and all of the embodiment, comparative examples 1 and 2 and the preparing example had cell proliferation effects. Specifically, the embodiment excelled the comparative examples 1 and 2 in cell proliferation effect and the black tea fermented liquid had the most excellent cell proliferation effect.

### Experiment Example 2

Confirmation of Free Radical Removal Effect by Black Tea Third Fermented Liquid of the Present Invention

It was confirmed the effects of the black tea fermented liquid prepared in embodiment 1 and comparative examples 1 and 2, and the black tea fermented extract prepared in preparing example 1 on free radical removal.

In order to confirm the effects on the free radical removal, the experiment had been performed as follows.

The examination, was performed by using DPPK (1,1-diphenyl-2picryl-hydrazine5 scheme (See Blois. M. S. Nature 181, 1190, 1958), where DPPH and quercetin of Sigma company were used. After methanol solution of 0.2 mM of 1 ml was inoculated with solutions of several, concentrations (5, 10, 20, 30 ppm) according to the embodiment, comparative examples 1 and 2, and the preparing example and then, agitated, they reacted for 10minutes at room temperature. Then, absorbance was measured at 570 nm. In this case, purified water was used instead of each sample in a bank test.

The variation of free radical was measured, by using following Formula 2 and the measurement results are shown in following Table 2.

$$Free Radical Effect ( \% ) = 100 - ( \frac{Reaction Absorbance Of Each Sample}{Absorbance o f Control Group} \times 100 ) \qquad [ Formula 2 ]$$

TABLE 2
Effect of free radical removal by black tea third
fermented liquid

| | Free radical effect (%) | | | |
|---|---|---|---|---|
| Concentration | | Comparative example | | Preparing |
| (%) | Embodiment | 1 | 2 | example |
| 0 | 0 | 0 | 0 | 0 |
| 5 | 49.1 | 19.6 | 15.3 | 18.8 |
| 10 | 87.6 | 41.6 | 33.6 | 45.6 |
| 30 | 99.1 | 79.3 | 82.1 | 81.9 |
| 50 | 100 | 91.6 | 95.4 | 96.8 |
| SC50 | 8.68 | 21.57 | 21.76 | 20.35 |

As shown in Table 2, it was confirmed that the free radical effects of the black tea fermented liquids prepared in comparative examples 1 and 2 are excellent more than that of the black tea extract prepared in the preparing example.

The SC50 represents harmful active oxygen scavenging activity and the concentration of the sample required to reduce harmful active oxygen of 50%.

### Experiment Example 3

Confirmation of Lipoxygenase Activity Prohibiting Effect by Black Tea Third Fermented Liquid of the Present Invention

The lipoxygenase inhibiting effects of the black tea fermented liquids prepared in embodiment 1 and comparative examples 1 and 2 and the black tea fermented extract prepared in preparing example 1 were confirmed.

In order to confirm the effects on the lipoxygenase activity inhibition, the experiment had been performed as fellows.

The examination was performed by using TBAS scheme, where linoleic acid, lipoxygenase and thiobabitulic acid of Sigma company were used. After linoleic acid of 1 mM and 1 ml was inoculated with solutions of 0.05 ml having several concentrations (5, 10, 20, 38, 40 ppm) according to the embodiment, comparative examples 1 and 2, and the preparing example and then lipoxygenase of 0.95 ml was added and agitated, they reacted for 10 minutes at 25° C. Then, after trichloroacetic acid of 0.5 ml and thiobarbiturlic acid of 1 ml were inoculated, it was heated for 10 minutes and cooled in iced water for 2˜3 minutes, so that the reaction was terminated. Butanol of 2 ml was added to the reaction terminated reaction solution and then, the solution was centrifuged for five minutes at 4,000 g. Then, absorbance was measured at 535 nm. In this case, purified water was used instead of each sample in a bank test.

The lipoxygenase activity inhibiting effect was obtained by using following Formula 3 and the results are shown in following Table 3.

$$Lipoxygenase Activity Inhibiting Effect ( \% ) = 100 - ( \frac{Reaction Absorbance Of Each Sample}{Absorbance o f Control Group} \times 100 ) \qquad [ Formula 3 ]$$

TABLE 3
Lipoxygenase activity prohibiting effect by black tea
third fermented liquid

| | Lipoxygenase activity prohibiting effect (%) | | | |
|---|---|---|---|---|
| Concentration | | Comparative example | | Preparing |
| (ppm) | Embodiment | 1 | 2 | example |
| 0 | 0 | 0 | 0 | 0 |
| 100 | 8.9 | 3.3 | 1.5 | 4.5 |
| 300 | 27.6 | 6.9 | 6.6 | 11.3 |
| 500 | 58.8 | 29.7 | 18.2 | 28.7 |
| 700 | 80.1 | 62.3 | 65.1 | 62.6 |
| 900 | 98.2 | 88.7 | 80.6 | 85.4 |
| SC50 | 455.45 | 586.83 | 619.13 | 584.99 |

As shown in Table 3, it was confirmed that the lipoxygenase activity prohibiting effect of the black tea fermented liquid prepared in the embodiment is excellent more than those of the black tea fermented liquids in comparative examples 1 and 2 and the black tea extract prepared in the preparing example.

### Experiment Example 4

### Confirmation of Hyaluroridase Activity Prohibiting Effect by Black Tea Third Fermented Liquid of the Present Invention

The hyaluronidase inhibiting effects of the black tea fermented liquids prepared in embodiment 1 and comparative examples 1 and 2 and the black tea extract prepared in preparing example 1 were confirmed.

In order to confirm the effects on the hyaluronidase activity inhibition, the experiment had been performed as follows.

According to a scheme to which Morgan-Bison scheme is applied, by using the final enzyme activity of hyaluronidase of 400NF unit/ml, and compound 43/80 buffer solution (0.1 mg/ml), the hyaluronidase activity was measured based on inhibiting reaction of an active stage of inactive hyaluronidase. The sample solution was obtained by dissolving a sample with a buffer and a buffer was used as the control group instead of sample solution. In addition, instead of enzyme solution, the buffer was used as each blank and green tea extract was used as the control group.

The hyaluronidase activity inhibiting effect was obtained by using following Formula 4 and the results are shown in following Table 4.

$$Hyaluronidase Activity Inhibiting Effect ( \% ) = 100 - ( \frac{Reaction Absorbance Of Each Sample}{Absorbance o f Control Group} \times 100 ) \qquad [ Formula 4 ]$$

TABLE 4
Hyaluronidase activity prohibiting effect by black tea
third fermented liquid

| | Hyaluronidase activity inhibiting effect (%) | | | |
|---|---|---|---|---|
| Concentration | | Comparative example | | Preparing |
| (%) | Embodiment | 1 | 2 | example |
| 0 | 0 | 0 | 0 | 0 |
| 0.5 | 23.4 | 7.6 | 6.6 | 8.9 |
| 1 | 41.9 | 19.8 | 15.4 | 23.5 |
| 3 | 81.2 | 41.6 | 36.1 | 40.1 |
| 7 | 98.6 | 79.3 | 69.7 | 79.3 |
| 10 | 100 | 91.8 | 88.7 | 95.2 |
| SC50 | 2.38 | 4.66 | 5.16 | 4.53 |

As shown In Table 4, it was confirmed that the hyaluronidase activity prohibiting effect of the black tea fermented liquid prepared in the embodiment is excellent more than those of the black tea fermented liquids in comparative examples 1 and 2 and the black tea extract prepared in the preparing example.

### Experiment Example 5

Irritant Relaxation Clinical Evaluation of Cosmetic Composition Including Black Tea Fermented Liquid According to the Present Invention

To perform irritant relaxation clinical evaluation of cosmetic composition including black tea fermented liquid of the present invention, the cosmetic composition (treatment example) having dried powder of black tea third fermented liquid according to the composition information denoted in following Table 5 and the cosmetic composition (comparative example) having no black tea third fermented liquid were prepared. The irritant relaxation clinical evaluation was performed by using the cosmetic compositions (treatment and comparative examples).

TABLE 5
Cosmetic composition for irritant relaxation clinical evaluation

| | | Treatment | Comparative |
|---|---|---|---|
| No. | Raw material | example | example |
| 1 | Powder of dried black tea third | 3.0 | — |
| | fermented liquid (embodiment) | | |
| 2 | Stearic acid | 2.0 | 2.0 |
| 3 | Cetyl alcohol | 2.0 | 2.0 |
| 4 | Glyceryl monostearate | 2.0 | 2.0 |
| 5 | Polyoxyethylene sorbitan monostearate | 0.5 | 0.5 |
| 6 | Sorbitan sesquioleate | 0.5 | 0.5 |
| 7 | Glyceryl stearate/glyceryl | 1.0 | 1.0 |
| | stearate/polyoxyethylene stearate | | |
| 8 | Wax | 1.0 | 1.0 |
| 9 | Flowing paraffin | 4.0 | 4.0 |
| 10 | Squalane | 4.0 | 4.0 |
| 11 | Caprylic/capric tryglycerides | 4.0 | 4.0 |
| 12 | Carboxyvinylpolymer | 0.3 | 0.3 |
| 13 | Butylene glycol | 5.0 | 5.0 |
| 14 | Glycerin | 3.0 | 3.0 |
| 15 | Triethanolamine | 0.5 | 0.5 |
| 16 | Purified water | Residual | Residual |
| 17 | Water | — | 3.0 |

To prepare the cosmetic composition of the treatment example, Nos. 12, 13, 14 and 16 were mixed and agitated and the mixture was heated at a temperature in the range of 80˜85° C., Then, after the heated mixture was supplied to a preparing unit, an emulsifier was operated. After Nos. 2, 3, 4, 5, 6, 7, 8, 9, 10 and 11 were heated at a temperature in the range of 80˜85° C. to be dissolved, No. 15 was supplied and agitated. Then, the mixture was supplied to the preparing unit to be emulsified. When the emulsifying was completed, the composition was agitated by using an agitator and cooled to 35° C. Then, after No. 1 was supplied and cooled to 25° C., the composition was aged.

To prepare the cosmetic composition of the comparative example, Nos. 12, 13, 14 and 16 were mixed and agitated and the mixture was heated at a temperature in the range of 80˜85° C. Then, after the heated mixture was supplied to a preparing unit, an emulsifier was operated. After Nos. 2, 3, 4, 5, 6, 7, 8, 9, 10 and 11 were heated at a temperature in the range of 80˜85° C. to be dissolved, No. 15 was supplied and agitated. Then, the mixture was supplied to the preparing unit to be emulsified. When the emulsifying was completed, the composition was agitated by using an agitator and cooled to 35° C. Then, after No. 17 was supplied and cooled to 25° C., the composition, was aged.

The irritant relaxation effect of the prepared cosmetic composition was confirmed through a simple clinical evaluation.

To evaluate the irritant relaxation effect of the cosmetic composition according to the treatment example, the following experiment was conducted on 50 woman applicants. To measure skin irritants of the applicants conduct under the same condition, the experiment portion was maintained in clean and dried state and after the skin was stabilized at an isothermal-isohumidity place (20° C., relative humidity 40˜60%) for at least 30 minutes, the experiment was conducted. After the portion of a nose is depilated by using a disposable razor, the treatment example was coated on the left side portion and the comparative example was coated on the right side portion based on a philtrum. After 20 minutes was elapsed thereafter, the skin irritant evaluation was conducted through surveys. The self measurement was conducted through self-survey evaluations of the applicants based on the contents of following Table 7, and the skin irritants of the treatment example and comparative example were compared with each other,

TABLE 6
Skin irritant evaluation reference

| 0 | 1 | 2 | 3 |
|---|---|---|---|
| Non- | Little | Normal | Irritant |
| irritant | | | |

TABLE 7
Skin irritant evaluation item and questionnaire

| | | | |
|---|---|---|---|
| Erythema | Edema | Itching | Prickling |

The survey results are shown in Table 8.

TABLE 8

Classification

| | Erythema | Edema | Itching | Prickling |
|---|---|---|---|---|
| Treatment | 0 | 0 | 0.6 | 0.4 |
| example | | | | |
| Comparative | 0 | 0 | 1.9 | 1.7 |
| example | | | | |

As shown in Table 8, it was confirmed that the cosmetics of the treatment example containing the black tea third fermented liquid (embodiment) have a very excellent irritant relaxation effect as compared with the cosmetics (comparative example) prepared, by adjusting water instead of the extract.

As described above, cosmetics of several, formulations containing the black tea third fermented liquid, which has very excellent cell proliferation, free radical removal, lipoxygenase activity prohibiting, hyaluroridase activity prohibiting and skin irritant relaxation effects, may be prepared.

Hereinafter, methods of preparing cosmetics of several, formulations containing the black tea third fermented liquid will be described,

### Formulation Example 1

Preparing of Cosmetic Water Containing Black Tea Third Fermented Liquid According to the Present Invention

A formulation example of cosmetic water (skin lotion) containing dried powder of black tea fermented liquid according to an embodiment is as following Table 9.

TABLE 9
Composition of cosmetic water containing black tea third
fermented liquid

| | | Content |
|---|---|---|
| No. | Raw material | (weight %) |
| 1 | Dried powder of black tea third fermented | 1.0 |
| | liquid (embodiment) | |
| 2 | Glucerin | 3.0 |
| 3 | Butylene glycol | 2.0 |
| 4 | Propylene glycol | 2.0 |
| 5 | Polyoxyethylene hydrogenated caster oil | 1.0 |
| 6 | Ethanol | 10.0 |
| 7 | Triethanolamine | 0.1 |
| 8 | Antiseptic | Trace |
| 9 | Pigment | Trace |
| 10 | Perfume | Trace |
| 11 | Purified water | Residual |

To prepare the cosmetic water, after Nos. 2, 3, 4 and 8were sequentially added to No. 11 denoted in Table 9, the mixture was agitated and dissolved. After No. 5 was heated to about 60° C. to be dissolved, No. 10 was supplied and dissolved, and then, No. 11 is added thereto. Lastly, after Nos. 1, 6, 7 and 9 were added thereto, it was sufficiently agitated and aged.

### Formulation Example 2

Preparing of Nutrition Lotion Containing Black Tea Third Fermented Liquid According to the Present Invention

The formulation example of the nutrition lotion among cosmetics containing dried powder of back tea third fermented liquid is as following Table 10.

TABLE 10
Composition of nutrition lotion containing black tea third
fermented liquid

| | | Content |
|---|---|---|
| No. | Raw material | (weight %) |
| 1 | Dried powder of black tea third | 2.0 |
| | fermented liquid (embodiment) | |
| 2 | Bee was | 1.0 |
| 3 | Polysorbate 60 | 1.5 |
| 4 | Sorbitan sesquioleate | 0.5 |
| 5 | Flowing paraffin | 10.0 |
| 6 | Sorbitan stearate | 1.0 |
| 7 | Lipophilic glyceryl monostearate | 0.5 |
| 8 | Stearic acid | 1.5 |
| 9 | Glyceryl stearate/PEG-400 stearate | 1.0 |
| 10 | Propylene glycol | 3.0 |
| 11 | Carboxypolymer | 0.1 |
| 12 | Triethanolamine | 0.2 |
| 13 | Antiseptic | Trace |
| 14 | Pigment | Trace |
| 15 | Perfume | Trace |
| 16 | Purified water | Residual |

To prepare the nutrition lotion, after Nos. 10, 11, 13 and 16 denoted in Table 10 were mixed and agitate, the mixture was heated at a temperature in the range of 80˜85° C. Then, after the heated mixture was supplied to the preparing unit, an emulsifier was operated, Nos. 2, 3, 4, 5, 6, 7, 8, 9 and 12were heated at a temperature in the range of 80˜85° C. to be dissolved and then, emulsified. When the emulsifying was s completed, after the emulsion was cooled to 50° C. while the emulsion was agitated by using agitator, No. 15 was supplied and cooled to 45° C. and then. No. 1 is added thereto at 35° C. and then, it was cooled to 25° C. and aged.

### Formulation Example 3

Preparing of Nutrition Cream Containing Black Tea Fermented Liquid According to the Present Invention

The formulation example of the nutrition cream among cosmetics containing dried powder of back tea third fermented liquid is as following Table 11.

TABLE 11

| | | Content |
|---|---|---|
| No. | Raw material | (weight %) |
| 1 | Dried powder of black tea third fermented | 3.0 |
| | liquid (embodiment) | |
| 2 | Stearic acid | 2.0 |
| 3 | Cetyl alcohol | 2.0 |
| 4 | Glyceryl monostearate | 2.0 |
| 5 | Polyoxyethylene sorbitan monostearate | 0.5 |
| 6 | Sorbitan sesquioleate | 0.5 |
| 7 | Glyceryl stearate/glyceryl | 1.0 |
| | stearate/polyoxyethylene atearate | |
| 8 | Wax | 1.0 |
| 9 | Flowing paraffin | 4.0 |
| 10 | Squalane | 4.0 |
| 11 | Caprylic/capric tryglycerides | 4.0 |
| 12 | Carboxyvinylpolymer | 0.2 |
| 13 | Butylene glycol | 5.0 |
| 14 | Glycerin | 3.0 |
| 15 | Triethanolamine | 0.5 |
| 16 | Purified water | [symbol] |

Composition of Nutrition Cream Containing Black Tea Fermented Liquid

To prepare the nutrition lotion, after Nos. 12, 13, 14 and 16 denoted in Table 11 were mixed and agitate, the mixture was heated at a temperature in the range of 80˜85° C. Then, after the heated mixture was supplied to the preparing unit, an emulsifier was operated. Nos. 2, 3, 4, 5, 6, 7, 8, 9, 10 and 11were heated at a temperature in the range of 80˜85° C. to he dissolved and then, No, 15 was added thereto and agitated. Then, it was supplied to the preparing unit and emulsified. When the emulsifying was completed, after the emulsion was cooled to 35° C. while the emulsion was agitated by using the agitator, No. 1 is added thereto and cooled to 25° C. Then, it was aged.

### Formulation Example 4

Preparing of Essence Containing Black Tea Fermented Liquid According to the Present Invention

The formulation example of the essence among cosmetics containing dried powder of back tea third fermented liquid is as following Table 12.

TABLE 12
Composition of essence containing black tea fermented liquid

| | | Contend |
|---|---|---|
| No | Raw material | (weight %) |
| 1 | Dried powder of black tea third fermented liquid | 3.0 |
| 2 | Sitosterol | 1.7 |
| 3 | Polyglyceryl-2 oleylether | 1.5 |
| 4 | Ceramide | 0.7 |
| 5 | Steareth-4 | 1.2 |
| 6 | Cholesterol | 1.5 |
| 7 | Dicetylphosphate | 0.4 |
| 8 | Concentrated glucerin | 5.0 |
| 9 | Macadamia oil | 15.0 |
| 10 | Carboxyvinylpolymer | 0.2 |
| 11 | Xanthan gum | 0.2 |
| 12 | Antiseptic | Trace |
| 13 | Perfume | Trace |
| 14 | Purified water | Residual |

To prepare the essence, Nos. 2, 3, 4, 5 and 6 were homogenized at a constant temperature and the homogenized material will be referred to as non-ion based amphipathic lipid. After the non-ion based amphipathic lipid was mixed with Nos. 1, 7, 8 and 14 and homogenized at a constant temperature, the non-ion based amphipathic lipid passed through a microfluidizer. Then, after No. 9 was slowly inoculated at a constant temperature and homogenized, it passed through the microfluidizer again. Next, No. 10, 11, 12 and 13 were supplied and distributed thereto to be stabilized and then, it was aged.

The cosmetic composition for improving anti-oxidation, anti-inflammation and atopic skin by using fermented liquid of black tea as an effective ingredient and the method of preparing the same described in this disclosure are for an illustrative purpose only, and the present invention is not limited thereto. Thus, it should be understood that numerous other modifications and embodiments can be devised by those skilled in the art within the spirit and scope of the present invention and they will fall within the scope of the present invention.

1. A cosmetic composition for improving anti-oxidation, anti-inflammation and atopic skin using fermented black tea liquid as an effective ingredient, wherein the cosmetic composition comprises the fermented black tea liquid obtained by primarily fermenting black tea with saccharomyces, secondarily fermenting the black tea with gluconacetobacter and tertiarily fermenting the black tea with bacillus.

2. A cosmetic composition for improving anti-oxidation, anti-inflammation and atopic skin using fermented black tea liquid as an effective ingredient, wherein the cosmetic composition comprises an extract of the fermented black tea liquid obtained by primarily fermenting black tea with saccharomyces, secondarily fermenting the black tea with gluconacetobacter and tertiarily fermenting the black tea with bacillus.

3. The cosmetic composition of claim 2, wherein the black tea is extracted by using one extract solvent selected from the group consisting of water, anhydrous or hydrous lower alcohol of hydrocarbon 1 to 4, acetone, ethyl acetate, butyl acetate, and 1,3-butylene glycol.

4. The cosmetic composition of claim 1, wherein a content of the fermented black tea liquid is in a range of 0.01 to 20.0% by weight based on a total weight of the cosmetic composition.

5. The cosmetic composition of claim 2, wherein a content of the extract of the fermented black tea liquid is in a range of 0.01 to 20.0% by weight based on a total weight of the cosmetic composition.

6. The cosmetic composition of claim 1, wherein the fermented black tea liquid has a selective effect of the anti-oxidation improvement, the anti-inflammation improvement and atopic skin improvement without a skin irritation side effect.

7. The cosmetic composition of claim 2, wherein the extract of the fermented black tea liquid has a selective effect of the anti-oxidation improvement, the anti-inflammation improvement and atopic skin improvement without a skin irritation side effect.

8. The cosmetic composition of claim 2, wherein the saccharmyces is one or at least two strains selected from the group consisting of saccharomyces bayanus, saccharomyces boulardii, saccharomyces cerevisiae, saccharomyces chevalieri, saccharomyces dairenensis, saccharomyces ellipsoideus, saccharomyces eubayanus, saccharomyces exiguus, saccharomyces florentinus and saccharomyces uvarum,
the gluconacetobacter is one or at least two strains selected from the group consisting of gluconacetobacter hansenii, gluconacetobacter intermedius, gluconacetobacter sacchari and gluconacetobacter xylinum, and
the bacillus is one or at least two strains selected from the group consisting of bacillus cereus, bacillus subtilis, bacillus licheniformis, bacillus megateriums, bacillus natto, bacillus citreus, bacillus circulans, bacillus mesentricus and bacillus pumilus.

9. A method of preparing a cosmetic composition for improving anti-oxidation, anti-inflammation and atopic skin by using fermented black tea liquid as an effective ingredient, the method comprises:
obtaining an extract of black tea (S10);
obtaining primary fermented liquid by inoculating saccharomyces as strain on the extract of the black tea (S20);
obtaining secondary fermented liquid by inoculating gluconacetobacter as strain on the primary fermented liquid (30); and
obtaining tertiary fermented liquid by inoculating bacillus as strain on the secondary fermented liquid (S40).

10. The method of claim 9, further comprising: obtaining an extract of the fermented black tea liquid through a hot-water extracting scheme after removing cultured bacteria from the tertiary fermented liquid (S50); and
obtaining the cosmetic composition by further adding stabilizer, vitamin, pigment, dye and perfume to the fermented black tea liquid of step S40 or to the extract of the fermented black tea liquid of step (S60).

11. The method of claim 9, wherein a content of the tertiary fermented black tea liquid obtained in step S40 is in a range of 0.01 % by weight to 20.0% by weight based on a total weight of the cosmetic composition.

12. The method of claim 10, wherein a content of the extract of the fermented black tea liquid is in a range of 0.01% by weight to 20.0% by weight based on a total weight of the cosmetic composition.

13. The cosmetic composition of claim 1, wherein the black tea is extracted by using one extract solvent selected from the group consisting of water, anhydrous or hydrous lower alcohol of hydrocarbon 1 to 4, acetone, ethyl acetate, butyl acetate, and 1,3-butylene glycol.

14. The cosmetic composition of claim 1, wherein the saccharomyces is one or at least two strains selected from the group consisting of saccharomyces bayanus, saccharomyces boulardii, saccharomyces cerevisiae, saccharomyces chevalieri, saccharomyces dairenensis, saccharomyces ellipsoideus, saccharomyces eubayanus, saccharomyces exiguus, saccharomyces florentinus and saccharomyces uvarum,
the gluconacetobacter is one or at least two strains selected from the group consisting of gluconacetobacter hansenii, gluconacetobacter intermedins, gluconacetobacter sacchari and gluconacetobacter xylinum, and
the bacillus is one or at least two strains selected from the group consisting of bacillus cereus, bacillus subtilis, bacillus licheniformis, bacillus megateriums, bacillus natto, bacillus citreus, bacillus circulans, bacillus mesentricus and bacillus pumilus.
